(19) United States
(12) Patent Application Publication
(10) Pub. No.: US 2005/0119239 A1
(43) Pub. Date: Jun. 2, 2005

(54) Pharmaceutical composition consisting of a beta-3-adrenoceptor agonist and an active substance which influences prostaglandin metabolism

(75) Inventors: Marion Wienrich, Weiterstadt (DE); Antje Howitz, Frankfurt (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/990,979

(22) Filed: Nov. 17, 2004

(30) Foreign Application Priority Data
Nov. 27, 2003 (DE) 103 56 112

(51) Int. Cl. A61K 31/60; A61K 31/415; A61K 31/24; A61K 31/192

(52) U.S. Cl. 514/165; 514/540; 514/406; 514/570

(57) ABSTRACT

This invention describes a new combination for the treatment of functional bladder disorders which comprises a beta-3-adrenoceptor agonist and an active substance which influences prostaglandin metabolism.

This invention describes a new active substance combination for the treatment of painful functional bladder disorders. According to the invention a pharmaceutical active substance combination is proposed consisting of at least one beta-3-adrenoceptor agonist and at least one active substance which influences prostaglandin metabolism.

Prior Art

The incidence of urinary incontinence is constantly increasing as a result of changes in the ageing statistics. Nevertheless those affected are for the most part still untreated or inadequately treated. Apart from the medical consequences such as chronic infections of the urinary passages, urinary incontinence for those affected is associated with a high psychological burden of suffering. It is estimated that 100 million older people are affected by urinary incontinence.

The lower urinary tract consists of the bladder, the urethra, the associated muscles, and the ligaments of the suspensory apparatus. The purpose of the bladder is to store the urine and evacuate it. The important factors for performing the storage function are not only the relaxation of the bladder muscle (detrusor muscle), but also the closure mechanisms provided by the neck of the bladder and the smooth muscle of the urethra and also by the cross-striated muscle of the urethra and the pelvic floor. During the emptying of the bladder (micturition) the detrusor muscle contracts while the urethra and pelvic floor relax and the sphincter muscle of the bladder opens. These processes require complex control by the parasympathetic, sympathetic, and somatic nervous system.

Functional bladder problems are a heterogeneous group of disorders which differ in their etiology, diagnosis, and therapy. In the standardizing recommendations of the International Continence Society (ICS) urinary incontinence is defined as involuntary loss of urine which is objectively detectable and constitutes a social and hygiene problem. Generally, urinary incontinence only occurs when there is an unintentional increase in the pressure in the bladder during the storage phase. This can happen as a result of unrestricted contractions of the detrusor muscle (urge incontinence) or failure of the urethral closure mechanism (stress incontinence).

According to the ICS definition, overactive bladder (OAB) is characterized by an irresistible imperative need to urinate, which may or may not be associated with urge incontinence, usually with increased frequency of micturition and nocturnal urination. Pathophysiologically, this complaint may be based on involuntary contractions during the filling phase, the cause of which may be neurogenic or non-neurogenic (idiopathic) in nature.

Urge incontinence is characterized by an irresistible urge to urinate and involuntary loss of urine.

Stress incontinence is characterized by the involuntary loss of urine which generally occurs at moments of elevated intra-abdominal pressure. This may occur for example when lifting, coughing, sneezing, running while at the same time there is no detrusor activity. Loss of urine takes place as the result of a variable combination of an insufficiency of the sphincter muscles of the bladder and the pelvic floor as well as anatomical defects in the suspensory apparatus. As a result the closure pressure of the urethra is too low and incontinence results. Pure stress incontinence often occurs in women, particularly if they have given birth. In men, this form of urinary incontinence is usually only observed after prostatectomies or other surgical interventions on the small pelvis.

In so-called mixed incontinence patients suffer from symptoms of both stress incontinence and urge incontinence. Once again, it is mainly women who are affected. For treating the various forms of functional bladder disorders, particularly stress incontinence, urge incontinence, mixed incontinence or overactive bladder (overactive bladder with or without urge incontinence), various therapeutic approaches are available.

For treating urge incontinence the WHO recommends anticholinergics (antimuscarinics). However, their use is limited because they are only moderately effective and particularly because they have serious side effects such as dryness of the mouth, accommodation disorders, constipation and central nervous effects (dizziness, fatigue, confusion).

Stress incontinence is treated primarily by conservative and surgical procedures. Up till now there has been no generally suitable drug therapy available. Alpha-adrenoceptor agonists such as pseudoephedrine and phenylpropanolamine have shown some effect, albeit very modest, in the treatment of low-grade stress incontinence. A disadvantage is that they have no selectivity for the urethral muscles and have numerous side effects such as hypertension, tachycardia, arrhythmia, sleep disorders, headaches, and tremors.

The treatment of mixed incontinence is a controversial subject of discussion and comprises combinations of invasive procedures for treating the stress incontinence component and drug therapies for treating the urge incontinence component.

Other forms of urinary incontinence are neurogenic incontinence, detrusor hyperreflexia or suburethral diverticulitis. Urinary tract infections may also result in urinary incontinence.

Since the mid-1995s it has been reported that selective beta-3-adrenoceptor-agonists are also promising in the treatment of urinary incontinence (EP 0 958 835). As the stimulation of beta-3-receptors is of exceptional importance for the relaxation of the detrusor muscle, the use of selective beta-3-adrenoceptors in patients with urge incontinence should result in the reduction or prevention of involuntary detrusor contractions during the urine storage phase. Tests with beta-3-adrenoceptor agonists indicate that they will be highly effective while being well tolerated. In addition, their activity should be restricted to the storage phase of the bladder and unimpeded emptying of the bladder should be guaranteed without any build-up of urine residues.

There are only limited options available for the treatment of overactive bladder as well. The less well-established forms of treatment also include drugs containing antimuscarinics as active substance.

Another interesting approach to the regulation of dysfunction of the bladder is drug intervention in prostaglandin biosynthesis. Prostaglandins appear to play a crucial role in the endogenous modulation of the micturition reflex. An increase in prostaglandin biosynthesis has also been observed in chronic bladder obstruction. In view of this and other observations, active substances which influence prostaglandin biosynthesis are steadily increasing in importance. A group of active substances which are representative in this respect are the non-steroidal anti-inflammatory compounds, NSAIDs for short. These compounds interact with the cyclooxygenase (COX) enzymes which occur as COX-1 and COX-2 and which are important in the synthesis of prostaglandins. Of particular importance is the enzyme COX-2 and with it, accordingly, COX-2 inhibitors for intervening in prostaglandin biosynthesis.

### SUMMARY OF THE INVENTION

Despite the many promising approaches and progress in the treatment of the various forms of urinary incontinence, which have been found to be causally complex and heterogeneous, the development of efficient and well-tolerated therapies remains a challenge.

The present invention sets out to contribute to the treatment of urinary incontinence. Preferably the invention is suitable for the treatment of stress incontinence, urge incontinence, mixed incontinence or overactive bladder (overactive bladder with or without urge incontinence).

It proposes a pharmaceutical composition which is intended to combine the advantages of the NSAIDs or cyclooxygenase inhibitors and those of the beta-3-adrenoceptor agonists in a manner which promotes the treatment of the underlying disease.

### DESCRIPTION OF THE INVENTION

According to the present invention a new pharmaceutical composition is provided which contains as active ingredients (a) an NSAID and/or cyclooxygenase inhibitor in a pharmaceutically effective amount and (b) at least one beta-3-adrenoceptor agonist in a pharmaceutically effective amount.

a) Active Components

In the description of the preferred embodiment certain terminology will be used hereinafter in the interests of clarity. This terminology should include the embodiment described and all technical equivalents which work in a similar manner for a similar purpose to achieve similar results. To the extent that any pharmaceutically active compound is disclosed or claimed, it is expressly intended that all active metabolites which are produced in vivo are included, and it is expressly intended that all enantiomers, diastereomers or tautomers are included, if the compound is capable of occurring in its enantiomeric, diastereomeric or tautomeric form. Obviously, the isomer which is pharmacologically most effective and most free from side effects is preferred. Also included are pharmacologically acceptable salts thereof. Examples of pharmaceutically active salts for each of the compounds which are the subject of this description include, without being restricted thereto, salts which are prepared from pharmaceutically acceptable acids or bases, including organic and inorganic acids and bases. If the preferred compound is basic, salts may be prepared from pharmaceutically acceptable acids. When selecting the most preferred salt, or to clarify whether a salt or the neutral compound is used, properties such as bioavailability, ease of manufacture, workability and shelf life are taken into consideration, inter alia. Suitable pharmaceutically acceptable acids include acetic acid, benzenesulphonic acid (besylate), benzoic acid, p-bromophenylsulphonic acid, camphorsulphonic acid, carbonic acid, citric acid, ethanesulphonic acid, fumaric acid, gluconic acid, glutamic acid, hydrobromic acid, hydrochloric acid, hydriodic acid, isethionic acid, lactic acid, maleic acid, malic acid, mandelic acid, methanesulphonic acid (mesylate), mucinic acid, nitric acid, oxalic acid, pamoic acid, pantothenic acid, phosphoric acid, succinic acid, sulphuric acid, tartaric acid, p-toluenesulphonic acid and the like. Examples of pharmaceutically acceptable salts include, without being restricted thereto, acetate, benzoate, hydroxybutyrate, bisulphate, bisulphite, bromide, butyne-1,4-dioate, caproate, chloride, chlorobenzoate, citrate, dihydrogen phosphate, dinitrobenzoate, fumarate, glycollate, heptanoate, hexyne-1,6-dioate, hydroxybenzoate, iodide, lactate, maleate, malonate, mandelate, metaphosphate, methanesulphonate, methoxybenzoate, methylbenzoate, monohydrogen phosphate, naphthalene-1-sulphonate, naphthalene-2-sulphonate, oxalate, phenylbutyrate, phenylproprionate, phosphate, phthalate, phenylacetate, propanesulphonate, propiolate, propionate, pyrophosphate, pyrosulphate, sebacate, suberate, succinate, sulphate, sulphite, sulphonate, tartrate, xylenesulphonate and the like.

Insofar as it is necessary for completeness, the methods of synthesis of the compounds for which the prior art is mentioned and the dosages thereof are expressly included by reference to the prior art mentioned at the corresponding point.

The COX-2 inhibitors are particularly preferred as cyclooxygenase inhibitors. Within the scope of the present specification the terms cyclooxygenase inhibitors or COX inhibitors are used in parallel. The same applies to COX-1 inhibitors or COX-2 inhibitors. By selective COX-2 inhibitors are meant compounds whose inhibitory effect on the enzyme COX-2 is greater than on the enzyme COX-1.

The following are preferred examples of suitable active substances from the group of NSAIDs with an effect on cyclooxygenase:
aa) acetylsalicylic acid, ab) indomethacin, ac) sulindac, ad) etodolac, ae) mefenamic acid, af) tolmetin, ag) ketorolac, ah) diclofenac, ai) ibuprofen, aj) naproxen, ak) fenoprofen, al) ketoprofen, am) oxaprozin, an) flurbiprofen, ao) nitroflurbiprofen, ap) piroxicam, aq) tenoxicam, ar) phenylbutazone, as) apazone, at) nimesulid and the pharmacologically acceptable salts thereof. Preferred examples are acetylsalicylic acid, ibuprofen, diclofenac, indomethacin, naproxen, flurbiprofen and/or nitroflurbiprofen.

Preferred examples of selective cyclooxygenase-2 inhibitors are listed as follows: au) meloxicam; av) RS-57067 (chemical name: 6-[[5-(4-chlorobenzoyl)-1,4-dimethyl-1H-pyrrol-2-yl]methyl]-3 (2H)-pyridazinone), aw) ABT-963, (chemical name: 2-(3,4-difluorophenyl)-4-(3-hydroxy-3-methylbutoxy)-5-[4-(methylsulphonyl)phenyl]-(9Cl)-3 (2H)-pyridazinone); ax) COX-189 (chemical name: 2-(2,4-dichloro-6-methyl-anilino)-4-ethylphenyl)-acetic acid); ay) NS-398, (chemical name: N-(2-cyclohexyl-4-nitrophenyl)methanesulphonamide; az) SD-8381 (chemical name: (S)-6,8-dichloro-2-(trifluoromethyl)-2H-1-benzopyran-3-carboxylic acid); ba) celecoxib; bb) valdecoxib; bc) deracoxib; bd) rofecoxib; be) etoricoxib (MK-663), bf) JTE-522 (chemical name: 5-methyl-3-phenyl-4-p-methylsulphonylphenyl-isoxazole). Meloxicam is preferred.

Each of the compounds listed above may be used for the treatment of urinary incontinence including at least one of the sub-indications mentioned hereinbefore, particularly stress incontinence, urge incontinence, mixed incontinence or overactive bladder.

The second component comprises one or more beta-3-adrenoreceptor agonists. This is preferably selected from the following group: 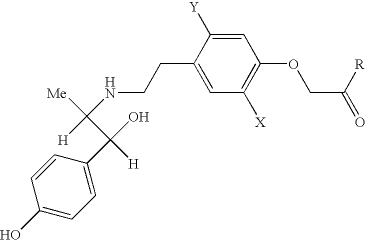
with
1) X═Br, Y═H, R═OH
2-[2-bromo-4-[2-[[(1S,2R)-2-hydroxy-2-(4-hydroxyphenyl)-1-methylethyl]amino]ethyl]phenoxy]acetic acid,
2) X═Cl, Y═H, R═OH
2-[2-chloro-4-[2-[[(1S,2R)-2-hydroxy-2-(4-hydroxyphenyl)-1-methylethyl]amino]ethyl]-phenoxy]acetic acid,
3) X═Y═Cl, R═OH
2-[2,5-dichloro-4-[2-[[(1S,2R)-2-hydroxy-2-(4-hydroxyphenyl)-1-methylethyl]amino]ethyl]phenoxy]acetic acid,
4) X═Y═H, R═OH
2-[4-[2-[[(1S,2R)-2-hydroxy-2-(4-hydroxyphenyl)-1-methylethyl]amino]ethyl]-2,5-dimethylphenoxy]acetic acid,
5) X═OH; Y═H; R═OH
2-[2-hydroxy-4-[2-[[(1S,2R)-2-hydroxy-2-(4-hydroxyphenyl)-1-methylethyl]amino]ethyl]-phenoxy]acetic acid,
6) X═Cl; Y═H, R═OEt
ethyl-2-[2-chloro-4-[2-[[(1S,2R)-2-hydroxy-2-(4-hydroxyphenyl)-1-methylethyl]amino]ethyl]phenoxy]acetate,
7) X═Cl; Y═Cl, R═OEt
ethyl-2-[2,5-dichloro-4-[2-[[(1S,2R)-2-hydroxy-2-(4-hydroxyphenyl)-1-methylethyl]amino]ethyl]phenoxy]acetate,
8) X=Me; Y=Me, R═OEt (−)-ethyl-2-[4-(2-{[(1S,2R)-2-hydroxy-2-(4-hydroxyphenyl)-1-methylethyl]amino}ethyl)-2,5-dimethylphenyloxy]acetate,
9) X=Me; Y=Me, R═OH
(−)-2-[4-(2-{[(1S,2R)-2-hydroxy-2-(4-hydroxyphenyl)-1-methylethyl]amino}ethyl)-2,5-dimethylphenyloxy]acetic acid,

Details of the abovementioned compounds 1 to 9 can be found in WO 00/02846 and its equivalent U.S. Pat. No. 6,538,152, which are incorporated herein by reference in their entirties.

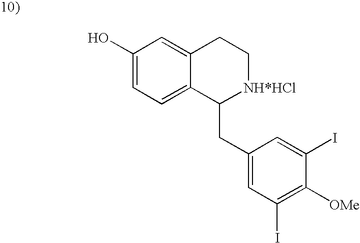

More detailed information on this substance can be found in J. Med. Chem. 44 (2001) 1456.

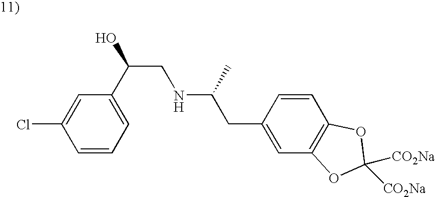

### Disodium-([R, R]-5-2-[[2-(3-chlorophenyl)-2-hydroxyethyl]-amino]propyl)-1,3-benzodioxole-2,2-dicarboxylate

More detailed information on this substance can be found in J. Med. Chem. 44 (2001) 1456 or in the Journal of Urology 165 (2001) 240.

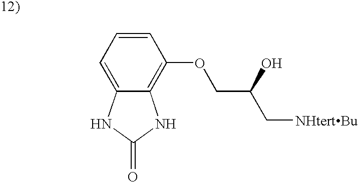

More detailed information on this substance, which is also known as CGP 12177A, can be found in Journal of Urology 165 (2001) 240 or in the J. Med. Chem. 44 (2001) 1456.

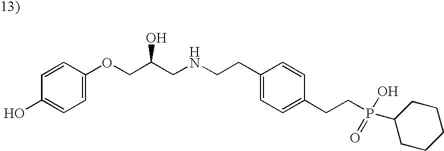

More detailed information on this substance, which is also known as SB 226552, can be found in J. Med. Chem. 44 (2001) 1456.

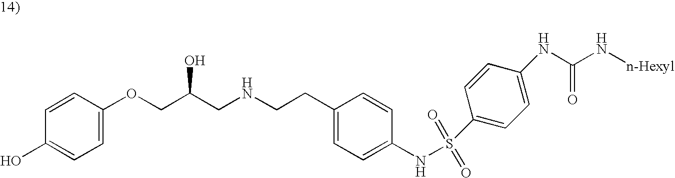

More detailed information on this substance, which is also known as L755507, can be found in J. Med. Chem. 44 (2001) 1456.

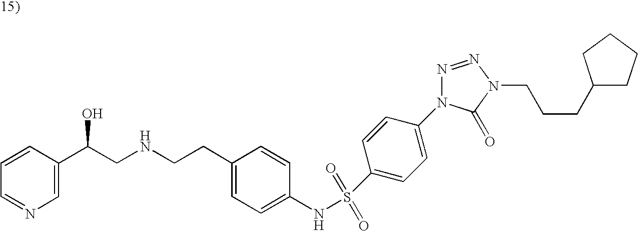

More detailed information on this substance, which is also known as L 770664, can be found in J. J. Med. Chem. 44 (2001) 1456.

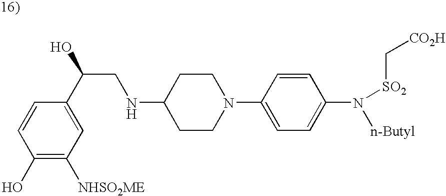

More detailed information on this substance can be found in J. Med. Chem. 44 (2001) 1456 or in the Bioorg. Med. Chem. Lett. 9 (2001) 2045. 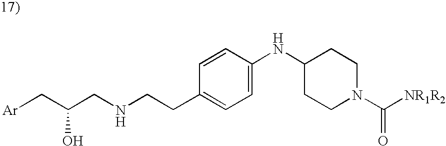
with
Ar=4-OHPh-O, R1=octyl, R2=H
Ar=4-OH,3-methylsulphonylamidophenyl-O, R1=2,5-diFbenzyl, R2=H
Ar=4-OH,3-methylsulphonylamidophenyl, R1=2,5-diFbenzyl, R2=H

More detailed information on these substances can be found in the Bioorg. Med. Chem. Lett. 11 (2000) 3123.

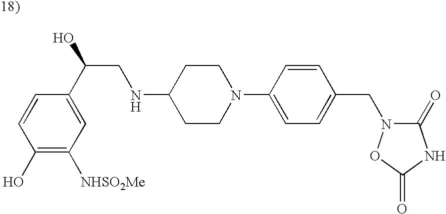

More detailed information on this substance can be found in the Bioorg. Med. Chem. Lett. 11 (2001) 981.

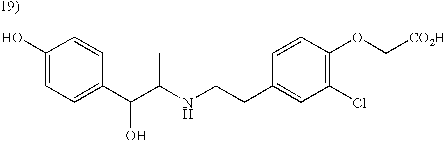

### 2-[2-chloro-4-(2-{[(1S, 2R)-2-hydroxy-2-(4-hydroxyphenyl)-1-methylethyl]amino}ethyl)phenoxy]acetic Acid

More detailed information on this substance can be found in the Med. Chem. 46 (2003) 105.

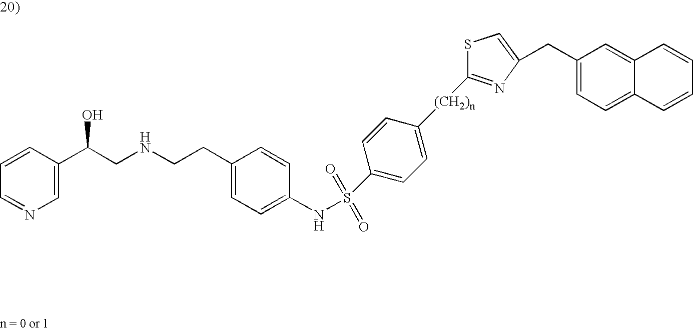

More detailed information on this substance can be found in the Bioorg. Med. Chem. Lett. 10 (2000) 1971.

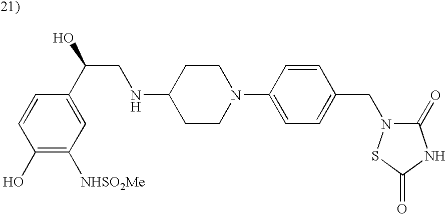

More detailed information on this substance can be found in the Bioorg. Med. Chem. Lett. 11 (2001) 757.

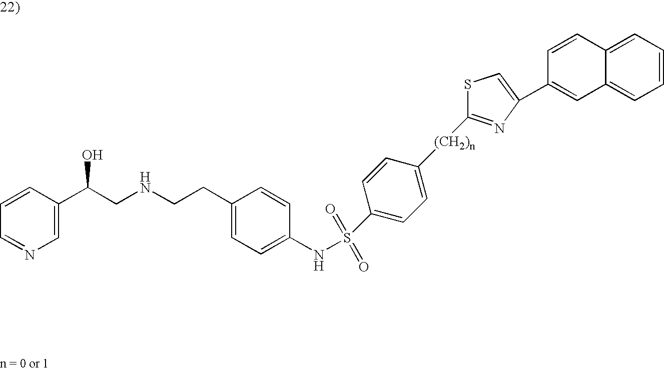

More detailed information on this substance can be found in the Bioor. Med. Chem. Lett. 10 (2000) 1971.

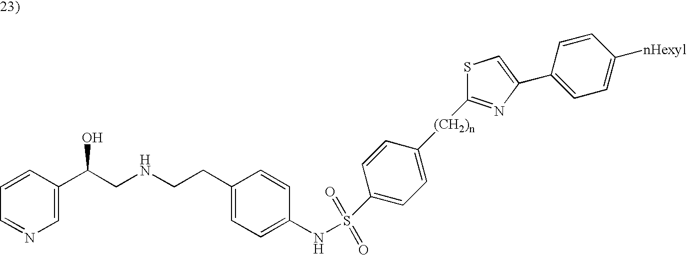

More detailed information on this substance can be found in the Bioor. Med. Chem. Lett. 10 (2000) 1971.

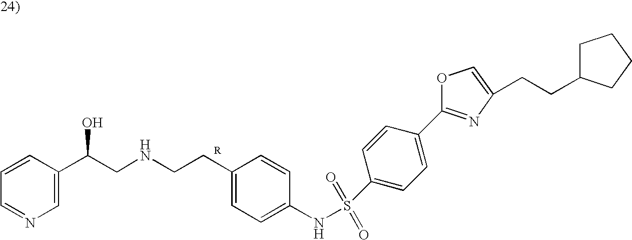

More detailed information on this substance can be found in the Bioorg. Med. Chem. Lett. 10 (2000) 1531. 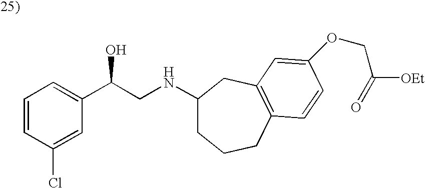
FK175
ethyl [R-(R*,S*)]-[[8-[[2-(3-chlorophenyl)-2-hydroxyethyl]amino]-6,7,8,9-tetrahydro-5H-benzocyclohepten-2-yl]oxy]-acetate, hydrochloride, 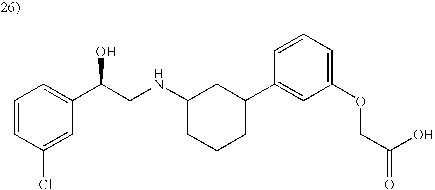
   GS-332
[1S-[1α,3β(S*)]]-3-[3-[[2-(3-chlorophenyl)-2-hydroxyethyl]amino]cyclohexyl]phenoxy]-acetic acid, monosodium salt,

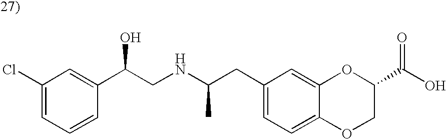

More detailed information on this compound which is also known as N-5984 can be found in the literature.
28) 2-(3-{[2-(3-chlorophenyl)-2R-hydroxyl-ethylamino]ethylamino}phenyl) furan-3-carboxylic acid. More detailed information on this compound can be found in the literature.
29) 2-(3-{[2-(3-chlorophenyl)-2R-hydroxyl-ethylamino]ethylamino}phenyl)thiophene-3-carboxylic acid. Information on this compound can be found in the literature.

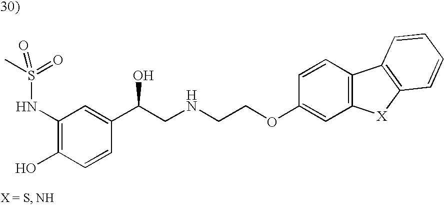

More detailed information on this compound also known as SB-418790 can be found in the literature.

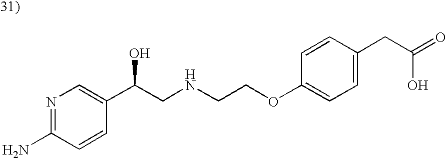

More detailed information on this compound also known as CP-331684 can be found in the literature.

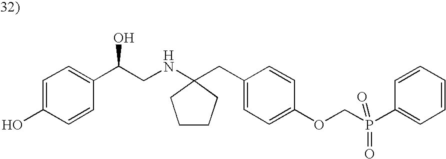

More detailed information on this compound also known as SB-251023 can be found in the literature.

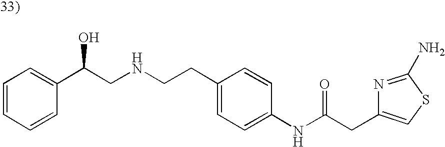

More detailed information on this compound, (R)-2-(2-aminothiazol-4-yl)-4′-[2-[2-(hydroxy-2-phenylethyl)amino]ethyl]acetanilide, can be found in the literature WO 03/037881.
34)
(S)-4-[2-hydroxy-3-[[2-[4-(5-carbamoyl-2-pyridyloxy)phenyl]-1,1-dimethyl-ethyl]amino]-propoxy]-carbazole (LY 377604).

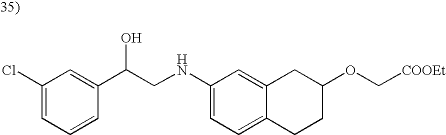

This compound is also known by the name SR 58611.

Beta-3-adrenoceptor agonists of the catecholamine type are preferred. Most preferred are:
(−)-ethyl-2-[4-(2-{[(1S,2R)-2-hydroxy-2-(4-hydroxyphenyl)-1-methylethyl]amino}ethyl)-2,5-dimethylphenyloxy]acetate,
(−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride,
(−)-2-[4-(2-{[(1S,2R)-2-hydroxy-2-(4-hydroxyphenyl)-1-methylethyl]amino}ethyl)-2,5-di-methylphenyloxy]acetic acid,
   or other pharmacologically acceptable salts thereof.

Particularly interesting examples of beta-3-adrenoceptor agonists are (−)-ethyl-2-[4-(2-{[(1S,2R)-2-hydroxy-2-(4-hydroxyphenyl)-1-methylethyl]amino}ethyl)-2,5-dimethylphenyloxy]acetate or (−)-2-[4-(2-{[(1S,2R)-2-hydroxy-2-(4-hydroxyphenyl)-1-methylethyl]-amino}ethyl)-2,5-dimethylphenyloxy]acetic acid, the enantiomers thereof, other diastereoisomers thereof and pharmacologically active salts thereof.

These compounds are disclosed in WO 00/02846 or WO 2003024916.

These last two compounds are represented by the following formula II, which should take precedence over the name given above, in the event of any inconsistencies: 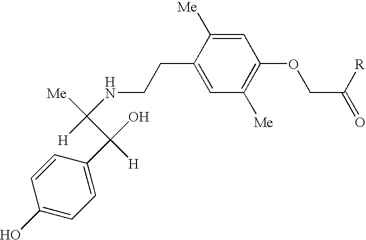
where R═O-ethyl: (−)-ethyl-2-[4-(2-{[(1S,2R)-2-hydroxy-2-(4-hydroxyphenyl)-1-methylethyl]amino}ethyl)-2,5-dimethylphenyloxy]acetate, preferably the monohydrate, where R═OH: (−)-2-[4-(2-{[(1S,2R)-2-hydroxy-2-(4-hydroxyphenyl)-1-methylethyl]-amino}ethyl)-2,5-dimethylphenyloxy]acetic acid.

Particularly preferred combinations contain each of the following possible combinations selected from (a) and (b):
(a) meloxicam, acetylsalicylic acid, diclofenac and/or ibuprofen and
(b) at least one of the following compounds: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid or any other pharmacologically acceptable salts thereof or any active metabolites thereof.

It is expressly pointed out that the invention includes every one of the following combinations: (aa, 1); (ab, 1); (ac, 1); (ad, 1); (ae, 1); (af, 1); (ag, 1); (ah, 1); (ai, 1); (aj, 1); (ak,1); (al, 1); (am,1); (an,1); (ao,1); (ap,1); (aq,1); (ar,1); (as,1); (at,1); (au,1); (av,1); (aw,1); (ax,1); (ay,1); (az,1); (ba, 1); (bb, 1); (bc, 1); (bd, 1); (be, 1); (bf, 1); (aa, 2); (ab, 2); (ac, 2); (ad, 2); (ae, 2); (af, 2); (ag, 2); (ah, 2); (ai, 2); (aj, 2); (ak,2); (al, 2); (am,2); (an,2); (ao,2); (ap,2); (aq,2); (ar,2); (as,2); (at,2); (au,2); (av,2); (aw,2); (ax,2); (ay,2); (az,2); (ba, 2); (bb, 2); (bc, 2); (bd, 2); (be, 2); (bf, 2); (aa, 3); (ab, 3); (ac, 3); (ad, 3); (ae, 3); (af, 3); (ag, 3); (ah, 3); (ai, 3); (aj, 3); (ak,3); (al, 3); (am,3); (an,3); (ao,3); (ap,3); (aq,3); (ar,3); (as,3); (at,3); (au,3); (av,3); (aw,3); (ax,3); (ay,3); (az,3); (ba, 3); (bb, 3); (bc, 3); (bd, 3); (be, 3); (bf, 3); (aa, 4); (ab, 4); (ac, 4); (ad, 4); (ae, 4); (af, 4); (ag, 4); (ah, 4); (ai, 4); (aj, 4); (ak,4); (al, 4); (am,4); (an,4); (ao,4); (ap,4); (aq,4); (ar,4); (as,4); (at,4); (au,4); (av,4); (aw,4); (ax,4); (ay,4); (az,4); (ba, 4); (bb, 4); (bc, 4); (bd, 4); (be, 4); (bf, 4); (aa, 5); (ab, 5); (ac, 5); (ad, 5); (ae, 5); (af, 5); (ag, 5); (ah, 5); (ai, 5); (aj, 5); (ak,5); (al, 5); (am,5); (an,5); (ao,5); (ap,5); (aq,5); (ar,5); (as,5); (at,5); (au,5); (av,5); (aw,5); (ax,5); (ay,5); (az,5); (ba, 5); (bb, 5); (bc, 5); (bd, 5); (be, 5); (bf, 5); (aa, 6); (ab, 6); (ac, 6); (ad, 6); (ae, 6); (af, 6); (ag, 6); (ah, 6); (ai, 6); (aj, 6); (ak,6); (al, 6); (am,6); (an,6); (ao,6); (ap,6); (aq,6); (ar,6); (as,6); (at,6); (au,6); (av,6); (aw,6); (ax,6); (ay,6); (az,6); (ba, 6); (bb, 6); (bc, 6); (bd, 6); (be, 6); (bf, 6); (aa, 7); (ab, 7); (ac, 7); (ad, 7); (ae, 7); (af, 7); (ag, 7); (ah, 7); (ai, 7); (aj, 7); (ak,7); (al, 7); (am,7); (an,7); (ao,7); (ap,7); (aq,7); (ar,7); (as,7); (at,7); (au,7); (av,7); (aw,7); (ax,7); (ay,7); (az,7); (ba, 7); (bb, 7); (bc, 7); (bd, 7); (be, 7); (bf, 7); (aa, 8); (ab, 8); (ac, 8); (ad, 8); (ae, 8); (af, 8); (ag, 8); (ah, 8); (ai, 8); (aj, 8); (ak,8); (al, 8); (am,8); (an,8); (ao,8); (ap,8); (aq,8); (ar,8); (as,8); (at,8); (au,8); (av,8); (aw,8); (ax,8); (ay,8); (az,8); (ba, 8); (bb, 8); (bc, 8); (bd, 8); (be, 8); (bf, 8); (aa, 9); (ab, 9); (ac, 9); (ad, 9); (ae, 9); (af, 9); (ag, 9); (ah, 9); (ai, 9); (aj, 9); (ak,9); (al, 9); (am,9); (an,9); (ao,9); (ap,9); (aq,9); (ar,9); (as,9); (at,9); (au,9); (av,9); (aw,9); (ax,9); (ay,9); (az,9); (ba, 9); (bb, 9); (bc, 9); (bd, 9); (be, 9); (bf, 9); (aa, 10); (ab, 10); (ac, 10); (ad, 10); (ae, 10); (af, 10); (ag, 10); (ah, 10); (ai, 10); (aj, 10); (ak,10); (al, 10); (am,10); (an,10); (ao,10); (ap,10); (aq,10); (ar,10); (as,10); (at,10); (au,10); (av,10); (aw,10); (ax,10); (ay,10); (az,10); (ba, 10); (bb, 10); (bc, 10); (bd, 10); (be, 10); (bf, 10); (aa, 11); (ab, 11); (ac, 11); (ad, 11); (ae, 11); (af, 11); (ag, 11); (ah, 11); (ai, 11); (aj, 11); (ak,11); (al, 11); (am, 11); (an, 11); (ao, 11); (ap, 11); (aq, 11); (ar, 11); (as, 11); (at, 11); (au, 11); (av,11); (aw,11); (ax,11); (ay,11); (az,11); (ba, 11); (bb, 11); (bc, 11); (bd, 11); (be, 11); (bf, 11); (aa, 12); (ab, 12); (ac, 12); (ad, 12); (ae, 12); (af, 12); (ag, 12); (ah, 12); (ai, 12); (aj, 12); (ak,12); (al, 12); (am,12); (an,12); (ao,12); (ap,12); (aq,12); (ar,12); (as,12); (at,12); (au,12); (av,12); (aw,12); (ax,12); (ay,12); (az,12); (ba, 12); (bb, 12); (bc, 12); (bd, 12); (be, 12); (bf, 12); (aa, 13); (ab, 13); (ac, 13); (ad, 13); (ae, 13); (af, 13); (ag, 13); (ah, 13); (ai, 13); (aj, 13); (ak,13); (al, 13); (am,13); (an,13); (ao,13); (ap,13); (aq,13); (ar,13); (as,13); (at, b 13); (au,13); (av,13); (aw,13); (ax, 13); (ay,13); (az,13); (ba, 13); (bb, 13); (bc, 13); (bd, 13); (be, 13); (bf, 13); (aa, 14); (ab, 14); (ac, 14); (ad, 14); (ae, 14); (af, 14); (ag, 14); (ah, 14); (ai, 14); (aj, 14); (ak,14); (al, 14); (am,14); (an,14); (ao,14); (ap,14); (aq,14); (ar,14); (as,14); (at,14); (au,14); (av,14); (aw,14); (ax,14); (ay,14); (az,14); (ba, 14); (bb, 14); (bc, 14); (bd, 14); (be, 14); (bf, 14); (aa, 15); (ab, 15); (ac, 15); (ad, 15); (ae, 15); (af, 15); (ag, 15); (ah, 15); (ai, 15); (aj, 15); (ak,15); (al, 15); (am,15); (an,15); (ao,15); (ap,15); (aq,15); (ar,15); (as,15); (at,15); (au,15); (av,15); (aw,15); (ax,15); (ay,15); (az,15); (ba, 15); (bb, 15); (bc, 15); (bd, 15); (be, 15); (bf, 15); (aa, 16); (ab, 16); (ac, 16); (ad, 16); (ae, 16); (af, 16); (ag, 16); (ah, 16); (ai, 16); (aj, 16); (ak,16); (al, 16); (am,16); (an,16); (ao,16); (ap,16); (aq,16); (ar,16); (as,16); (at,16); (au,16); (av,16); (aw,16); (ax,16); (ay,16); (az,16); (ba, 16); (bb, 16); (bc, 16); (bd, 16); (be, 16); (bf, 16); (aa, 17); (ab, 17); (ac, 17); (ad, 17); (ae, 17); (af, 17); (ag, 17); (ah, 17); (ai, 17); (aj, 17); (ak,17); (al, 17); (am,17); (an,17); (ao,17); (ap,17); (aq,17); (ar,17); (as,17); (at,17); (au,17); (av,17); (aw,17); (ax,17); (ay,17); (az,17); (ba, 17); (bb, 17); (bc, 17); (bd, 17); (be, 17); (bf, 17); (aa, 18); (ab, 18); (ac, 18); (ad, 18); (ae, 18); (af, 18); (ag, 18); (ah, 18); (ai, 18); (aj, 18); (ak,18); (al, 18); (am,18); (an,18); (ao,18); (ap,18); (aq,18); (ar,18); (as,18); (at,18); (au,18); (av,18); (aw,18); (ax,18); (ay,18); (az,18); (ba, 18); (bb, 18); (bc, 18); (bd, 18); (be, 18); (bf, 18); (aa, 19); (ab, 19); (ac, 19); (ad, 19); (ae, 19); (af, 19); (ag, 19); (ah, 19); (ai, 19); (aj, 19); (ak,19); (al, 19); (am,19); (an,19); (ao,19); (ap,19); (aq,19); (ar,19); (as,19); (at,19); (au,19); (av,19); (aw,19); (ax,19); (ay,19); (az,19); (ba, 19); (bb, 19); (bc, 19); (bd, 19); (be, 19); (bf, 19); (aa, 20); (ab, 20); (ac, 20); (ad, 20); (ae, 20); (af, 20); (ag, 20); (ah, 20); (ai, 20); (aj, 20); (ak,20); (al, 20); (am,20); (an,20); (ao,20); (ap,20); (aq,20); (ar,20); (as,20); (at,20); (au,20); (av,20); (aw,20); (ax,20); (ay,20); (az,20); (ba, 20); (bb, 20); (bc, 20); (bd, 20); (be, 20); (bf, 20); (aa, 21); (ab, 21); (ac, 21); (ad, 21); (ae, 21); (af, 21); (ag, 21); (ah, 21); (ai, 21); (aj, 21); (ak,21); (al, 21); (am,21); (an,21); (ao,21); (ap,21); (aq,21); (ar,21); (as,21); (at,21); (au,21); (av,21); (aw,21); (ax,21); (ay,21); (az,21); (ba, 21); (bb, 21); (bc, 21); (bd, 21); (be, 21); (bf, 21); (aa, 22); (ab, 22); (ac, 22); (ad, 22); (ae, 22); (af, 22); (ag, 22); (ah, 22); (ai, 22); (aj, 22); (ak,22); (al, 22); (am,22); (an,22); (ao,22); (ap,22); (aq,22); (ar,22); (as,22); (at,22); (au,22); (av,22); (aw,22); (ax,22); (ay,22); (az,22); (ba, 22); (bb, 22); (bc, 22); (bd, 22); (be, 22); (bf, 22); (aa, 23); (ab, 23); (ac, 23); (ad, 23); (ae, 23); (af, 23); (ag, 23); (ah, 23); (ai, 23); (aj, 23); (ak,23); (al, 23); (am,23); (an,23); (ao,23); (ap,23); (aq,23); (ar,23); (as,23); (at,23); (au,23); (av,23); (aw,23); (ax,23); (ay,23); (az,23); (ba, 23); (bb, 23); (bc, 23); (bd, 23); (be, 23); (bf, 23); (aa, 24); (ab, 24); (ac, 24); (ad, 24); (ae, 24); (af, 24); (ag, 24); (ah, 24); (ai, 24); (aj, 24); (ak,24); (al, 24); (am,24); (an,24); (ao,24); (ap,24); (aq,24); (ar,24); (as,24); (at,24); (au,24); (av,24); (aw,24); (ax,24); (ay,24); (az,24); (ba, 24); (bb, 24); (bc, 24); (bd, 24); (be, 24); (bf, 24); (aa, 25); (ab, 25); (ac, 25); (ad, 25); (ae, 25); (af, 25); (ag, 25); (ah, 25); (ai, 25); (aj, 25); (ak,25); (al, 25); (am,25); (an,25); (ao,25); (ap,25); (aq,25); (ar,25); (as,25); (at,25); (au,25); (av,25); (aw,25); (ax,25); (ay,25); (az,25); (ba, 25); (bb, 25); (bc, 25); (bd, 25); (be, 25); (bf, 25); (aa, 26); (ab, 26); (ac, 26); (ad, 26); (ae, 26); (af, 26); (ag, 26); (ah, 26); (ai, 26); (aj, 26); (ak,26); (al, 26); (am,26); (an,26); (ao,26); (ap,26); (aq,26); (ar,26); (as,26); (at,26); (au,26); (av,26); (aw,26); (ax,26); (ay,26); (az,26); (ba, 26); (bb, 26); (bc, 26); (bd, 26); (be, 26); (bf, 26); (aa, 27); (ab, 27); (ac, 27); (ad, 27); (ae, 27); (af, 27); (ag, 27); (ah, 27); (ai, 27); (aj, 27); (ak,27); (al, 27); (am,27); (an,27); (ao,27); (ap,27); (aq,27); (ar,27); (as,27); (at,27); (au,27); (av,27); (aw,27); (ax,27); (ay,27); (az,27); (ba, 27); (bb, 27); (bc, 27); (bd, 27); (be, 27); (bf, 27); (aa, 28); (ab, 28); (ac, 28); (ad, 28); (ae, 28); (af, 28); (ag, 28); (ah, 28); (ai, 28); (aj, 28); (ak,28); (al, 28); (am,28); (an,28); (ao,28); (ap,28); (aq,28); (ar,28); (as,28); (at,28); (au,28); (av,28); (aw,28); (ax,28); (ay,28); (az,28); (ba, 28); (bb, 28); (bc, 28); (bd, 28); (be, 28); (bf, 28); (aa, 29); (ab, 29); (ac, 29); (ad, 29); (ae, 29); (af, 29); (ag, 29); (ah, 29); (ai, 29); (aj, 29); (ak,29); (al, 29); (am,29); (an,29); (ao,29); (ap,29); (aq,29); (ar,29); (as,29); (at,29); (au,29); (av,29); (aw,29); (ax,29); (ay,29); (az,29); (ba, 29); (bb, 29); (bc, 29); (bd, 29); (be, 29); (bf, 29); (aa, 30); (ab, 30); (ac, 30); (ad, 30); (ae, 30); (af, 30); (ag, 30); (ah, 30); (ai, 30); (aj, 30); (ak,30); (al, 30); (am,30); (an,30); (ao,30); (ap,30); (aq,30); (ar,30); (as,30); (at,30); (au,30); (av,30); (aw,30); (ax,30); (ay,30); (az,30); (ba, 30); (bb, 30); (bc, 30); (bd, 30); (be, 30); (bf, 30); (aa, 31); (ab, 31); (ac, 31); (ad, 31); (ae, 31); (af, 31); (ag, 31); (ah, 31); (ai, 31); (aj, 31); (ak,31); (al, 31); (am,31); (an,31); (ao,31); (ap,31); (aq,31); (ar,31); (as,31); (at,31); (au,31); (av,31); (aw,31); (ax,31); (ay,31); (az,31); (ba, 31); (bb, 31); (bc, 31); (bd, 31); (be, 31); (bf, 31); (aa, 32); (ab, 32); (ac, 32); (ad, 32); (ae, 32); (af, 32); (ag, 32); (ah, 32); (ai, 32); (aj, 32); (ak,32); (al, 32); (am,32); (an,32); (ao,32); (ap,32); (aq,32); (ar,32); (as,32); (at,32); (au,32); (av,32); (aw,32); (ax,32); (ay,32); (az,32); (ba, 32); (bb, 32); (bc, 32); (bd, 32); (be, 32); (bf, 32); (aa, 33); (ab, 33); (ac, 33); (ad, 33); (ae, 33); (af, 33); (ag, 33); (ah, 33); (ai, 33); (aj, 33); (ak,33); (al, 33); (am,33); (an,33); (ao,33); (ap,33); (aq,33); (ar,33); (as,33); (at,33); (au,33); (av,33); (aw,33); (ax,33); (ay,33); (az,33); (ba, 33); (bb, 33); (bc, 33); (bd, 33); (be, 33); (bf, 33); (aa, 34); (ab, 34); (ac, 34); (ad, 34); (ae, 34); (af, 34); (ag, 34); (ah, 34); (ai, 34); (aj, 34); (ak,34); (al, 34); (am,34); (an,34); (ao,34); (ap,34); (aq,34); (ar,34); (as,34); (at,34); (au,34); (av,34); (aw,34); (ax,34); (ay,34); (az,34); (ba, 34); (bb, 34); (bc, 34); (bd, 34); (be, 34); (bf, 34); (aa, 35); (ab, 35); (ac, 35); (ad, 35); (ae, 35); (af, 35); (ag, 35); (ah, 35); (ai, 35); (aj, 35); (ak,35); (al, 35); (am,35); (an,35); (ao,35); (ap,35); (aq,35); (ar,35); (as,35); (at,35); (au,35); (av,35); (aw,35); (ax,35); (ay,35); (az,35); (ba, 35); (bb, 35); (bc, 35); (bd, 35); (be, 35); (bf, 35).

b) Dosage

In order to determine the optimum dose of the two active substances for urinary incontinence, various basic conditions have to be taken into consideration such as for example the age and body weight of the patient, the nature and stage of the disease and the potency of the compound. This is deemed to be within the capabilities of the skilled man, and the existing literature on the components can be consulted in order to arrive at the optimum dose. The doses specified relate to the dosage after the end of the adjustment phase.

The doses given hereinafter expressly include all the numerical values, both whole numbers and fractions, within the range specified. The data relate to adults. Pediatric doses may be lower.

Doses administered more than once a day or twice a day (e.g. 3, 4, 5 or 6 times a day) are also expressly included herein.

The preferred oral dose of the cyclooxygenase inhibitor in humans is 0.1 mg to 200 mg per day and kg body weight, preferably between 1 mg and 50 mg per day and kg body weight and most preferably between 1 mg and 10 mg per day and kg body weight.

The intravenous dose for each of the above-mentioned compounds may be lower than the oral dose by a factor 10, preferably by a factor 100.

In some cases a smaller amount may be sufficient while in other cases a larger total amount may be required.

The total daily dose may be taken in one go or in several portions depending on the treatment plan. The treatment plan may also prescribe intervals of longer than one day between the doses.

The choice of dosage for this first component (a) is the dose which provides relief for the patient.

The daily dose of the combination according to the invention desirably contains in the case of the active substance meloxicam as component (a) this in an amount from about 0.5 mg to about 50 mg. Preferably, each dose of the component contains about 1 to about 25 mg of the active substance.

For acetylsalicylic acid the preferred daily dose is 0,1 mg to 4000 mg, preferably 10 mg to 2000 mg.

For ibuprofen the preferred daily dose is 0.1 mg to 6000 mg, preferably 10 mg to 3000 mg.

For diclofenac, e.g. as diclofenac-sodium, the preferred daily dose is 0.1 mg to 500 mg, preferably 10 mg to 250 mg.

This dosage form enables the complete daily dose to be administered in half or whole doses, once or several times. The invention also expressly includes administration more than once a day or twice a day (e.g. 3, 4, 5, or 6 doses per day).

The doses and the treatment plan (i.e. one, two, three, or more doses per day) of the second component depend on the factors to which reference has already been made in conjunction with the choice of dosage for the first component.

The average daily dose for adults of the second component (beta-3-agonist) is about 1 mg to about 1000 mg, preferably 10 mg to about 750 mg per day, preferably 5 to 120 mg, more preferably 10 to 100 mg, administered in one or more doses. This dose is preferably administered orally. The intravenous dose is preferably lower than the oral dose by a factor 10, more preferably 100.

c) Formulations

The compositions of the present invention may conveniently be administered in a pharmaceutical composition which contains the active components in combination with a suitable carrier. Such pharmaceutical compositions may be prepared by methods and contain carriers which are well known in the art. Generally recognized textbooks are available to the skilled man for this purpose.

The compositions of the present invention may be administered parenterally (e.g. by intravenous, intraperitoneal, subcutaneous or intramuscular injection), topically, orally, intranasally, transdermally, rectally, by pulmonary or nasal inhalation, oral administration being particularly preferred. Of the oral formulations, those which are resistant to gastric juices are preferred. Therefore, capsules or tablets resistant to gastric juices are preferred, and in both cases this may be achieved with a coating which is resistant to gastric juices. The skilled man will find instructions for formulations resistant to gastric juices in the prior art.

Various formulating options are described below. The skilled man may choose a suitable formulation from them.

For oral therapeutic administration the composition according to the invention may be combined with one or more carriers and used in the form of tablets for swallowing, buccal tablets, sublingual tablets, sugar-coated tablets, sprays, powders, pastilles, coated tablets, granules, capsules, elixirs, suspensions, solutions, syrups, lozenges, chewing gums, foods and the like.

A powder may be prepared for example by grinding the particles of active substance to a suitable size.

Dilute powders may be prepared by finely grinding the powdered substance with a non-toxic carrier material such as lactose and delivering it as a powder. Other suitable carrier materials for this purpose are other carbohydrates such as starch or mannitol. These powders may optionally contain flavourings, preservatives, dispersing agents, colourings and other pharmacological adjuvants.

Capsules may be prepared from a powder of the kind described above or other powders, which are placed in a capsule, preferably a gelatine capsule, and the capsule is then sealed.

It is also possible for lubricants known from the prior art to be introduced into the capsule or used to seal the two parts of the capsule. The efficacy of a capsule when taken orally can be increased by the addition of disintegrating or solubilising substances such as, for example, carboxymethylcelluose, carboxymethylcellulose calcium, low-substituted hydroxypropylcellulose, calcium carbonate, sodium carbonate and other substances. The active substance may be present in the capsule not only as a solid but also in suspended form, for example in vegetable oil, polyethyleneglycol, or glycerol using surface-active substances, etc.

Tablets may be prepared by compressing the powdered mixture and then processing it into granules, for example. The tablets may contain various excipients such as e.g. starches, lactose, sucrose, glucose, sodium chloride, urea for tablets for dissolving or injecting, amylose, various types of cellulose as described above and others. Glycerol or starch may be used as a moisture retaining agent.

The disintegrants used may be, for example, starch, alginic acid, calcium alginate, pectic acid, powdered agar-agar, formaldehyde gelatine, calcium carbonate, sodium bicarbonate, magnesium peroxide and amylose.

Anti-disintegrants or solution retardants which may be used include, for example, sucrose, stearin, solid paraffin (preferably with a melting point in the range from 50-52° C.), cocoa butter and hydrogenated fats.

Other disintegrants may be: corn starch, potato starch, alginic acid and the like.

Suitable absorption accelerators include, inter alia, quaternary ammonium compounds, sodium lauryl sulphate and saponins.

Ether may be used, for example, as a binder distributor and cetyl alcohol, glycerol monostearate, starch, corn starch, lactose, wetting agents (e.g. aerosol OT, Pluronics, Tweens), gum tragacanth, gum arabic, gelatine and others may be used as hydrophilising agents or disintegration accelerators.

Sucrose, fructose, lactose, or aspartame may be used as sweeteners while peppermint, wintergreen oil, cherry flavouring etc may be used as flavouring agents.

The following may also be generally used as additional excipients: Aerosil, Aerosol OT ethylcellulose, Amberlite resin, XE-88, Amijel, Amisterol, amylose, Avicel microcrystalline-cellulose, bentonite, calcium sulphate, Carbowax 4000 and 6000, carrageen, castor wax, cellulose, microcrystalline cellulose, crospovidone, dextrane, dextrin, dicalcium phosphate, pharmaceutical tablet base, kaolin, lactose (USP), lactosil, magnesium stearate, mannitol, granular mannitol N. F. methylcellulose, Miglyol 812 neutral oil, powdered milk, powdered sugar, nal-tab, nepol-amylose, Pöfizer crystalline sorbitol, plasdone, polyethyleneglycols, polyvinylacetate phthalate, polyvinylpyrrolidone, Precirol, neat's foot oil (hydrogenated), melting tablet base, silicone, stabiline, Starx 1500, syloid, Waldhof tablet base, tablettol, talcum cetylatum and stearatum, Tego metal soaps, fructose and tylose. The tabletting excipient K (M25) is particularly suitable, and also complies with the requirements of the following pharmacopoeias: DAB, Ph, Eur, BP and NF.

Other excipients which may be used can be found in the Examples, but other excipients known from the prior art may also be used.

The tablets may be produced by direct compression, for example.

It is also possible to prepare other formulations for oral administration such as solutions, syrups, elixirs etc. If desired the compound may be micro-encapsulated.

Parenteral administration may be achieved by dissolving the compound in a liquid and injecting it by subcutaneous, intramuscular or intravenous route. Suitable solvents include, for example, water or oily media.

In order to prepare suppositories the compound may be formulated with low-melting and water-soluble or water-insoluble materials such as polyethylene glycol, cocoa butter, higher esters (for example moerysthyl, palmitate) or mixtures thereof.

The above list is provided solely by way of example and a skilled man might consider other excipients.

Various other materials may be provided as coatings or for modifying the physical form of the solid dosage units in some other way. For example, tablets, pills or capsules may be coated with gelatine, wax, shellac or sugar and the like. As already mentioned, formulations resistant to gastric juices are preferred for the oral preparations. Therefore, gastric juice-resistant coatings are preferred for tablets or capsules. In the case of a syrup or elixir, sucrose or fructose may be used as the sweetener, methyl- and propylparaben may be present as preservatives and a colouring and a flavouring agent such as cherry or orange flavour may also be present.

The excipients mentioned above are not restricted to the use of the formulation in connection with which they have been mentioned but may also be applied to the other formulations.

Naturally, any material used in the preparations of any of these dosage units must be pharmaceutically acceptable and substantially non-toxic in the amounts used. In addition, the active components may be incorporated in preparations with delayed release and devices which, without being restricted thereto, include those based on osmotic pressures, in order to achieve the desired release profile. One-a-day formulations for each of the active components are particularly included.

Compositions and preparations of this kind should contain at least 0.001% of active compound. The percentage of the compositions and preparations may naturally vary and may appropriately make up between 0.1 and about 100% of the weight of a given dosage unit. The quantity of active compound in therapeutically useful compositions of this kind is such that an effective dose is present.

The composition according to the invention which contains the two active components may be administered in the same physical form or at the same time in accordance with the dosages described above and in the administration carriers described above. The dosages for each active component may be measured separately and may be administered as a single combined dose or separately. They may be given at the same time or at different times provided that both active ingredients come to act in the patient at some time over a 24 hour period. It is preferable if the two components act in such a way as to achieve an effect which is better than the individual activity in each case. Simultaneous or coincident administration means that the patient takes one drug within about five minutes of taking the other drug. For ease of handling it is preferable to use formulations in which the two drugs are given to the patient close together and typically at the same time.

d) Indications

The pharmaceutical composition may preferably be used to treat or prevent, inter alia, each of the syndromes mentioned below, as an individual syndrome and in conjunction with another of the syndromes mentioned, without being restricted thereto: urinary incontinence, particularly stress incontinence, urge incontinence, mixed incontinence or overactive bladder of neurogenic or non-neurogenic origin, neurogenic incontinence, detrusor hyperreflexia, suburethral diverticulitis, urinary tract infections and further sub-indications thereof.

Thus, the invention includes both those syndromes whose cause is dysfunction or disease of an organ and those which can be attributed to diseases or disorders of the central nervous system. Accordingly, every treatment of bladder function disorder, particularly urinary incontinence of all kinds, is taken into account by the present invention.

Thus, a further embodiment of the present invention comprises using the composition according to the invention to prepare a drug for treating or preventing any of the indications of bladder dysfunction mentioned in the preceding paragraph.

The above diseases or disorders are treated by administering a therapeutically effective amount of the composition according to the invention to a mammal. In most cases this is a human being but the treatment of farm animals (e.g. cattle) and domestic animals (e.g. dogs, cats and horses) is also expressly covered. For use in veterinary medicine the dosages used may be different from those specified herein.

It is expected that the new composition will provide rapid relief for those suffering from the above diseases and disorders with a minimum amount of harmful side effects.

### e) EXAMPLES

The invention is illustrated by the following non-restrictive Examples.

Particularly preferred combinations are:
a) meloxicam and (−)-ethyl-2-[4-(2-{[(1S,2R)-2-hydroxy-2-(4-hydroxyphenyl)-1-methyl-ethyl]amino}ethyl)-2,5-dimethylphenyloxy]acetate.
b) meloxicam and (−)-ethyl-2-[4-(2-{[(1S,2R)-2-hydroxy-2-(4-hydroxyphenyl)-1-methyl-ethyl]amino}ethyl)-2,5-dimethylphenyloxy]acetate-monohydrochloride.
c) meloxicam and (−)-2-[4-(2-{[(1S,2R)-2-hydroxy-2-(4-hydroxyphenyl)-1-methylethyl]-amino}ethyl)-2,5-dimethylphenyloxy]acetic acid.
d) meloxicam and (−)-2-[4-(2-{[(1S,2R)-2-hydroxy-2-(4-hydroxyphenyl)-1-methylethyl]-amino}ethyl)-2,5-dimethylphenyloxy]acetic acid-monohydrochloride.
e) ibuprofen and (−)-ethyl-2-[4-(2-{[(1S,2R)-2-hydroxy-2-(4-hydroxyphenyl)-1-methyl-ethyl]amino}ethyl)-2,5-dimethylphenyloxy]acetate.
f) ibuprofen and (−)-ethyl-2-[4-(2-{[(1S,2R)-2-hydroxy-2-(4-hydroxyphenyl)-1-methyl-ethyl]amino}ethyl)-2,5-dimethylphenyloxy]acetate-monohydrochloride.
g) ibuprofen and (−)-2-[4-(2-{[(1S,2R)-2-hydroxy-2-(4-hydroxyphenyl)-1-methylethyl]-amino}ethyl)-2,5-dimethylphenyloxy]acetic acid.
h) ibuprofen and (−)-2-[4-(2-{[(1S,2R)-2-hydroxy-2-(4-hydroxyphenyl)-1-methylethyl]-amino}ethyl)-2,5-dimethylphenyloxy]acetic acid-monohydrochloride.
j) diclofenac-sodium and (−)-ethyl-2-[4-(2-{[(1S,2R)-2-hydroxy-2-(4-hydroxyphenyl)-1-methylethyl]amino}ethyl)-2,5-dimethylphenyloxy]acetate.
k) diclofenac-sodium and (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride.
l) diclofenac-sodium and (−)-2-[4-(2-{[(1S,2R)-2-hydroxy-2-(4-hydroxyphenyl)-1-methylethyl]amino}ethyl)-2,5-dimethylphenyloxy]acetic acid.
m) diclofenac-sodium and (−)-2-[4-(2-{[(1S,2R)-2-hydroxy-2-(4-hydroxyphenyl)-1-methylethyl]amino}ethyl)-2,5-dimethylphenyloxy]acetic acid-monohydrochloride.
n) acetylsalicylic acid and (−)-ethyl-2-[4-(2-{[(1S,2R)-2-hydroxy-2-(4-hydroxyphenyl)-1-methylethyl]amino}ethyl)-2,5-dimethylphenyloxy]acetate.
o) acetylsalicylic acid and (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride.
p) acetylsalicylic acid and (−)-2-[4-(2-{[(1S,2R)-2-hydroxy-2-(4-hydroxyphenyl)-1-methylethyl]amino}ethyl)-2,5-dimethylphenyloxy]acetic acid.
q) acetylsalicylic acid and (−)-2-[4-(2-{[(1S,2R)-2-hydroxy-2-(4-hydroxyphenyl)-1-methylethyl]amino}ethyl)-2,5-dimethylphenyloxy]acetic acid-monohydrochloride.

Now that the invention has been described in detail with reference to the preferred embodiments, it is clear that modifications and alterations are possible without departing from the scope of the attached claims.

### Example 1

### Composition Containing (−)-ethyl-2-[4-(2-{[(1S,2R)-2-hydroxy-2-(4-hydroxyphenyl)-1-methylethyl]amino}ethyl)-2,5-dimethylphenyloxy]acetate and acetylsalicylic acid-tablet 40 mg/500 mg

| Ingredients | mg/tablet |
|---|---|
| (−)-ethyl-2-[4-(2-{[(1S, 2R)-2-hydroxy-2-(4-hydroxyphenyl)- | 43.640 |
| 1-methylethyl]amino}ethyl)-2,5-dimethylphenyloxy]acetate- | |
| monohydrochloride | |
| acetylsalicylic acid | 500.000 |
| microcrystalline cellulose | 102.360 |
| maize starch | 34.000 |
| total weight of tablet | 680.000 |

### Example 2

### Composition Containing (−)-ethyl-2-[4-(2-{[(1S,2R)-2-hydroxy-2-(4-hydroxyphenyl)-1-methylethyl]amino}ethyl)-2,5-dimethylphenyloxy]acetate and meloxicam-tablet 80 mg/7.5 mg

| Ingredients | mg/tablet |
|---|---|
| (−)-ethyl-2-[4-(2-{[(1S, 2R)-2-hydroxy-2-(4-hydroxyphenyl)- | 87.280 |
| 1-methylethyl]amino}ethyl)-2,5-dimethylphenyloxy]acetate- | |
| monohydrochloride | |
| meloxicam | 7.500 |
| lactose monohydrate | 30.220 |
| microcrystalline cellulose | 80.000 |
| povidone | 15.000 |
| purified water | (q.s.) |
| crospovidone | 22.500 |
| silicon dioxide | 5.000 |
| magnesium stearate | 2.500 |
| total weight of tablet | 250.000 |

### Example 3

Composition Containing (−)-ethyl-2-[4-(2-{[(1S,2R)-2-hydroxy-2-(4-hydroxyphenyl)-1-methylethyl]amino}ethyl)-2,5-dimethylphenyloxy]acetate and ibuprofen—Film-coated tablet 40 mg/200 mg

| | |
|---|---|
| Ingredients | mg/tablet |

Core

| | |
|---|---|
| (−)-ethyl-2-[4-(2-{[(1S, 2R)-2-hydroxy-2-(4-hydroxyphenyl)- | 43.640 |
| 1-methylethyl]amino}ethyl)-2,5-dimethylphenyloxy]acetate- | |
| monohydrochloride | |
| ibuprofen | 200.000 |
| lactose monohydrate | 118.860 |
| microcrystalline cellulose | 80.000 |
| sodium carboxymethyl starch | 20.000 |
| hydroxypropylmethylcellulose | 15.000 |
| stearinpalmitic acid | 7.500 |
| silicon dioxide | 5.000 |
| purified water | (q.s.) |

Film coating

| | |
|---|---|
| hydroxypropylmethylcellulose | 6.000 |
| propyleneglycol | 0.750 |
| titanium dioxide | 1.500 |
| talc | 1.750 |
| purified water | (q.s.) |
| total weight of film-coated tablet | 500.000 |

### Example 4

### Composition Containing (−)-ethyl-2-[4-(2-{[(1S,2R)-2-hydroxy-2-(4-hydroxyphenyl)-1-methylethyl]amino}ethyl)-2,5-dimethylphenyloxy]acetate and diclofenac-sodium—gastric juice-resistant tablet 80 mg/50 mg

| | |
|---|---|
| Ingredients | mg/tablet |

Core

| | |
|---|---|
| (−)-ethyl-2-[4-(2-{[(1S, 2R)-2-hydroxy-2-(4-hydroxyphenyl)- | 87.280 |
| 1-methylethyl]amino}ethyl)-2,5-dimethylphenyloxy]acetate- | |
| monohydrochloride | |
| diclofenac-sodium | 50.000 |
| calcium hydrogen phosphate | 100.000 |
| lactose | 140.220 |
| cellulose | 50.000 |
| maize starch | 50.000 |
| sodium carboxymethyl starch | 15.000 |
| magnesium stearate | 2.500 |
| silicon dioxide | 5.000 |
| purified water | (q.s.) |

Gastric juice-resistant coating

| | |
|---|---|
| methylacrylic acid-ethylacrylate-copolymer | 38.800 |
| triethylcitrate | 4.000 |
| Polysorbate 80 | 4.000 |
| glycerol monostearate | 1.200 |
| titanium dioxide | 2.000 |
| purified water | (q.s.) |
| total weight of coated tablet | 550.000 |

1. Pharmaceutical composition comprising: (a) a first active agent comprising a pharmaceutically effective amount of one or more NSAIDs or cyclooxygenase inhibitors, or a pharmacologically acceptable salt, enantiomer, diastereomer, tautomer, or metabolite thereof, and (b) a second active agent comprising a pharmaceutically effective amount of one or more beta-3-adrenoceptor agonists or a pharmacologically acceptable salt, enantiomer, diastereomer, tautomer, or metabolite thereof.

2. Pharmaceutical composition according to claim 1, wherein the first active agent is selected from the group consisting of: acetylsalicylic acid, indomethacin, sulindac, etodolac, mefenamic acid, tolmetin, ketorolac, diclofenac, ibuprofen, naproxen, fenoprofen, ketoprofen, oxaprozin, flurbiprofen, nitroflurbiprofen, piroxicam, tenoxicam, phenylbutazone, apazone, nimesulid, meloxicam, RS-57067, ABT-963, COX-189, NS-398, SD-8381, celecoxib, valdecoxib, deracoxib, rofecoxib, etoricoxib, JTE-522, and pharmacologically acceptable salts, enantiomers, diastereomers, tautomers, and metabolites thereof, and mixtures thereof.

3. Pharmaceutical composition according to claim 1, wherein the first active agent is selected from the group consisting of: meloxicam, acetylsalicylic acid, ibuprofen, diclofenac, and pharmacologically acceptable salts, enantiomers, diastereomers, tautomers, and metabolites thereof, and mixtures thereof.

4. Pharmaceutical composition according to one of claim 1, wherein the second active agent is selected from the group consisting of:
(−)-ethyl-2-[4-(2-{[(1S,2R)-2-hydroxy-2-(4-hydroxyphenyl)-1-methylethyl]-amino}-ethyl)-2,5-dimethylphenyloxy]acetate,
(−)-ethyl-2-[4-(2-{[(1S,2R)-2-hydroxy-2-(4-hydroxyphenyl)-1-methylethyl]-amino}-ethyl)-2,5-dimethylphenyloxy]acetate-monohydrochloride, and
(−)-2-[4-(2-{[(1S, 2R)-2-hydroxy-2-(4-hydroxyphenyl)-1-methylethyl]amino}ethyl)-2,5-di-methylphenyloxy]acetic acid,
and pharmacologically acceptable salts, enantiomers, diastereomers, tautomers, and metabolites thereof, and mixtures thereof.

5. Pharmaceutical composition according to one of claim 1, wherein:
the first active agent is selected from the group consisting of: meloxicam, acetylsalicylic acid, ibuprofen, diclofenac, and pharmacologically acceptable salts, enantiomers, diastereomers, tautomers, and metabolites thereof, and mixtures thereof, and
the second active agent is selected from the group consisting of: (−)-ethyl-2-[4-(2-{[(1S,2R)-2-hydroxy-2-(4-hydroxyphenyl)-1-methylethyl]-amino}-ethyl)-2,5-dimethylphenyloxy]acetate, (−)-ethyl-2-[4-(2-{[(1S,2R)-2-hydroxy-2-(4-hydroxyphenyl)-1-methylethyl]-amino}-ethyl)-2,5-dimethylphenyloxy]acetate-monohydrochloride, and (−)-2-[4-(2-{[(1S,2R)-2-hydroxy-2-(4-hydroxyphenyl)-1-methylethyl]amino}ethyl)-2,5-di-methylphenyloxy]acetic acid, and pharmacologically acceptable salts, enantiomers, diastereomers, tautomers, and metabolites thereof, and mixtures thereof.

6. Pharmaceutical composition according to claim 1, comprising about 0.5 mg to about 500 mg of the first active agent, and about 10 mg to about 750 mg of the second active agent.

7. Pharmaceutical composition according to any one of claim 1, wherein the first and second active agents are formulated in the same pharmaceutical form.

8. Pharmaceutical composition according to any one of claim 1, wherein the first and second active agents are formulated in different pharmaceutical forms.

9. Pharmaceutical composition according to claim 1 adapted for rectal, topical, oral, sublingual, intranasal, transdermal, or parenteral administration.

10. Pharmaceutical composition according to claim 1 adapted for the simultaneous administration of the the first and second active agents.

11. Pharmaceutical composition according to claim 1, wherein the release of at least one of the first and second active agents is at least partially delayed after administration.

12. Pharmaceutical composition according to claim 1, wherein at least one of the first and second active agents is at least partially released immediately upon administration.

13. Method of treating a functional bladder disorder in a mammal comprising administering to the mammal a pharmaceutical composition comprising: (a) a first active agent comprising a pharmaceutically effective amount of one or more NSAIDs or cyclooxygenase inhibitors, or a pharmacologically acceptable salt, enantiomer, diastereomer, tautomer, or metabolite thereof, and (b) a second active agent comprising a pharmaceutically effective amount of one or more beta-3-adrenoceptor agonists or a pharmacologically acceptable salt, enantiomer, diastereomer, tautomer, or metabolite thereof.

14. Method according to claim 13, wherein the first active agent is selected from the group consisting of: acetylsalicylic acid, indomethacin, sulindac, etodolac, mefenamic acid, tolmetin, ketorolac, diclofenac, ibuprofen, naproxen, fenoprofen, ketoprofen, oxaprozin, flurbiprofen, nitroflurbiprofen, piroxicam, tenoxicam, phenylbutazone, apazone, nimesulid, meloxicam, RS-57067, ABT-963, COX-189, NS-398, SD-8381, celecoxib, valdecoxib, deracoxib, rofecoxib, etoricoxib, JTE-522, and pharmacologically acceptable salts, enantiomers, diastereomers, tautomers, and metabolites thereof, and mixtures thereof.

15. Method according to claim 13, wherein the first active agent is selected from the group consisting of: meloxicam, acetylsalicylic acid, ibuprofen, diclofenac, and pharmacologically acceptable salts, enantiomers, diastereomers, tautomers, and metabolites thereof, and mixtures thereof.

16. Method according to one of claim 13, wherein the second active agent is selected from the group consisting of:
(−)-ethyl-2-[4-(2-{[(1S,2R)-2-hydroxy-2-(4-hydroxyphenyl)-1-methylethyl]-amino}-ethyl)-2,5-dimethylphenyloxy]acetate,
(−)-ethyl-2-[4-(2-{[(1S,2R)-2-hydroxy-2-(4-hydroxyphenyl)-1-methylethyl]-amino}-ethyl)-2,5-dimethylphenyloxy]acetate-monohydrochloride, and
(−)-2-[4-(2-{[(1S,2R)-2-hydroxy-2-(4-hydroxyphenyl)-1-methylethyl]amino}ethyl)-2,5-di-methylphenyloxy]acetic acid, and pharmacologically acceptable salts, enantiomers, diastereomers, tautomers, and metabolites thereof, and mixtures thereof.

17. Method according to one of claim 13, wherein:
the first active agent is selected from the group consisting of: meloxicam, acetylsalicylic acid, ibuprofen, diclofenac, and pharmacologically acceptable salts, enantiomers, diastereomers, tautomers, and metabolites thereof, and mixtures thereof, and
the second active agent is selected from the group consisting of: (−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-di-methylphenyloxy]acetic acid, and pharmacologically acceptable salts, enantiomers, diastereomers, tautomers, and metabolites thereof, and mixtures thereof.

18. Method according to claim 13, comprising about 0.5 mg to about 500 mg of the first active agent, and about 10 mg to about 750 mg of component the second active agent.

19. Method according to claim 13, wherein the functional bladder disorder is urinary incontinence or overactive bladder or a disease or disorder of the central nervous system that is related to functional bladder disorders.

20. Method according to claim 13, wherein the functional bladder disorder is urinary incontinence, urge incontinence, stress incontinence, mixed incontinence, other forms of urinary incontinence and/or overactive bladder.
